(19) United States
(12) Patent Application Publication
(10) Pub. No.: US 2007/0117176 A1
(43) Pub. Date: May 24, 2007

(54) Process for the extraction of atelopeptide collagen from a collagenous source by microbial treatment

(76) Inventors: Srinivasan Aishwarya, Chennai (IN); Viswanaathan Shashirekha, Chennai (IN); Praveen Kumar Sehgal, Chennai (IN)

Correspondence Address: MEYERTONS, HOOD, KIVLIN, KOWERT & GOETZEL, P.C., 700 LAVACA, SUITE 800, AUSTIN, TX, 78701, US

(21) Appl. No.: 11/394,140

(22) Filed: Mar. 30, 2006

(30) Foreign Application Priority Data
Nov. 18, 2005 (IN) 3133/DEL/2005

(51) Int. Cl. A61K 38/17; C12P 21/06; C07K 14/78

(52) U.S. Cl. 435/68.1; 530/356

(57) ABSTRACT

Bovine collagen is freed of non-collagen proteins, glycosaminoglycans and lipids by microbial treatment to yield a product, which is undenatured fibre of high tensile strength. The microbial extraction technique ensures degradation of non-collagenous components with the help of the protease combination produced, leaving the collagenous matter intact due to the absence of collagenase in the secreted enzymes. The resulting atelopeptide collagen has largely monomeric triple helical conformation. This process results in the formation of regularly ordered fibres of collagen possessing a rope-like structure. It is soluble in dilute acidic aqueous solutions. The collagen is rendered non-immunogenic by the removal of certain terminal peptide chains. The non-cytotoxic fibres can be fabricated into various physical forms for biomedical applications.

### FIELD OF THE INVENTION

The present invention relates to a process for the extraction of atelopeptide collagen from a collagenous source by microbial treatment.

More particularly, the invention relates to an extraction process to derive collagen fibres of high tensile strength from collagenous tissue. It is envisaged to have enormous potential applications in the pharmaceutical industry for preparing cost effective collagenous biomaterials useful for medical applications. Moreover, the atelopeptide collagen extracted by this process can be used as a suture material for surgical applications. It may also be molded in diverse shapes, ensuring its use as an ideal carrier, wound cover or drug delivery scaffold, physically cut fibres with any sharp instrument including a sterile scalpel to suit the dimensions of a punctual plug for medical applications.

### BACKGROUND OF THE INVENTION

The non-helical regions, saccharides, mucopolysaccharide associated with collagen responsible for immunogenicity and other contaminating proteins are removed by microbial treatment to obtain “atelopeptide” collagen.

The importance of collagen as a biomaterial rests largely on the fact that it is a natural material of low immunogenicity and is therefore recognized by the body as a normal constituent rather than a foreign matter as cited by Friess (European Journal of Pharmaceutics and Biopharmaceutics Vol. 45, 113-116, 1998). Collagen has been produced in a range of physical forms such as sheets, tubes, sponges, powder and fleece to be utilized in medical process as reported by Srivastava et al. (Biomaterials, Vol. 11, 155-161, 1990).

Although collagen is ubiquitous in the mammalian body, those tissues rich in fibrous collagen such as skin and tendon are generally used as starting materials to generate collagen for use in implants, wound dressings or drug delivery systems as reported by Friess (European Journal of Pharmaceutics and Biopharmaceutics, Vol. 45, 113-116, 1998).

Fibrous collagen possesses high tensile strength and its high degree of natural crosslinking makes it easier to purify without degradation. Tendon collagen is relatively purer and more resistant to decomposition in view of the inherent cross-links, which are very firm. The helical structure of collagen provides it resistance to proteolysis and also masks the antigenic deteminants. Collagen is a natural substance for cell adhesion and the major tensile load-bearing component of the musculo-skeletal system. Because of the inherent tensile properties, it has application in the manufacture of implantable prostheses and in the preparation of living tissue equivalents.

For use as a biomaterial, collagen is recovered in the undenatured form i.e. with a little or no damage to the basic rigid triple helical structure. The undenatured collagen in solubilised or fibril form from crude collagen source eg. skin, tendon, hide etc. is obtained by acid, base, salt and enzyme extraction.

Reference may be made to Friess (European Journal of Pharmaceutics and Biopharmaceutics Vol. 45, 113-116, 1998) wherein isolation of neutral salt soluble, acid soluble and alkali treated collagen has been described. Soluble collagen is purified by precipitation after pH, salt concentration or temperature adjustment.

Reference may be made to Kemp et al. (U.S. Pat. No. 5,106,949), who subjected common digital extensor tendon to acid extraction followed by precipitation of the resulting extract to obtain pure collagen.

The extraction of native or intact collagen fibrils in the cold with dilute organic acids leads to the solubilization of undenatured collagen molecules or aggregates of them. In fact, acid solutions of soluble collagen have been the primary systems available for study by physical, chemical and electron optical techniques. The extraction of rat tail tendon collagen with successive fresh portions of citrate buffer at pH 3.4 until no further acid soluble collagen was extracted has also been described. The residual collagen was extracted with acetate buffer. Some native soluble collagen could be extracted from intact collagens by the action of various salts at pH value near neutrality. This fraction was termed neutral salt-soluble collagen as cited by Ramachandran (Treatise on collagen, Vol. 1, Academic Press Inc., New York, 379-380, 1967).

Gunasekaran (U.S. Pat. No. 6,548,077) has reported the major limitations associated with some acid treatments involving acid solubilization of bovine tendon collagen to produce a collagen suspension. This suspension is then either dialyzed or precipitated in saline, resulting in an amorphous precipitate containing non-fibrillary denatured collagen. Collagen prepared according to this method is generally not directly suitable for medical purposes, as it lacks tensile strength in moist media and has little resistance against enzymatic degradation when applied to living tissue.

The conventional method to obtain collagen in fibre form is to subject the collagenous source to a range of mechanical operations like sheathing, slicing, homogenizing in specialized “micro cut” machines to separate individual tendon fibres, as reported by Miyata et al. (U.S. Pat. No. 4,271,070) and washing them repeatedly with dilute salt solutions prior to enzyme treatment. The major limitation associated with this process is that the fibre is disintegrated into small fibrils, whereby the strength of the fibre reduces significantly. As reported by Luck et al. (U.S. Pat. No. 4,488,911), the enzyme extraction method is preferred for the preparation of solubilised collagen, regardless of the nature of the tissue as the methodology leads to increased yields and higher purity collagen. Collagen typically of bovine origin is extracted from tissue in dilute aqueous acid and then digested with a protease such as pepsin, trypsin or pronase to remove the telopeptides from the ends of the collagen molecules. Atelopeptide collagen fibres are conventionally reconstituted from collagen in solution (CIS) by raising the pH of the solution. Smestad et al. (U.S. Pat. No. 4,582,640 assigned to Collagen Corporation) describes a crosslinked form of atelopeptide fibrillar collagen. Hide or skin is cut. into pieces of workable size and slurried in acidified water in the presence of pepsin, trypsin, pronase or proctase. After treatment the enzyme is inactivated by raising the pH of the solution and later precipitating it at pH 7.0. An injectable suspension of this crosslinked material is available commercially from Collagen Corporation under the trademark ZYPLAST. RTM. The product is prepacked in a syringe in the same manner as the ZYDERM. RTM product. While this commercial material is remarkably effective, it may shrink in volume after implantation primarily due to absorption of its fluid component by the body. Thus if volume constancy, sometimes called “persistency”, is essential, an additional injection or injections of supplemental implant material is required. One disadvantage of treatment with pepsin is that the collagen preparations may be partially degraded.

The fibre contact lenses prepared by Miyata et al. (U.S. Pat. No. 4,268,131) is by enzyme extraction of collagen. Tendon fiber dispersion after micro-cut treatment is washed in sodium chloride solution and the collagen is collected by centrifugation. Washing with sodium chloride is repeated two or three times. Finally the collagen is washed with water to remove sodium chloride. The washed collagen is treated with pancreatin to remove telopeptides, saccharides and proteins other than collagen. The enzyme-treated collagen is collected by centrifugation and washed with 5% sodium chloride and finally with water. Then the collagen is treated with ethanol to remove water and lipid, then with 1:1 mixture of ethanol and ether to remove lipid or fat. Ethanol-ether extraction is carried out at room temperature with stirring for 1 day. The powdered collagen is swollen in citric acid or hydrochloric acid solution at pH 2-3. This transparent tendon fibril is stabilized by gamma ray irradiation or by chemical crosslinking and converted, after pouring into a mold into a collagen gel lens.

Gunasekaran (U.S. Pat. No. 6,548,077) provided a method for purification of collagen by subjecting the enzyme extracted collagen solution to two papain treatments, followed by treating the resulting solution with reducing and delipidating agents.

Further the alterations in pH during enzyme treatment causes salt formation, which have to be thoroughly removed from the collagen. Disposal of the wash water also posses a problem due to the presence of salt.

Furthermore, the enzyme-extracted collagen produces tissue equivalents, which are undesirably weak for certain applications involving substantial mechanical handling of the tissue equivalent.

Another limitation associated with the conventional process of preparing collagen is that this process results in the loss of tensile strength of the innate collagenous material.

Yet another limitation associated with the conventional process of preparing collagen is that the process is time consuming involving several processing steps.

Still another limitation associated with the conventional process of preparing collagen is that the process is not cost effective for large-scale production.

In order to overcome the above-mentioned limitations, a process of extracting fibre collagen using a microbe was developed. Tendon is a structure whose function is to transmit tensile loads between muscle and bone. Morphologically, it has been described as a complex composite material consisting of collagen fibrils embedded in a hydrated matrix of proteoglycans as reported by Haut (Journal of Biomechanics, Vol. 19, No. 11, 951-955, 1986). Collagen fibers are the most abundant fibre found in connective tissue. Their inelasticity and molecular configuration provide collagen fibres with a tensile strength that is greater than steel. Thus, collagen provides a combination of flexibility and strength to the tissues in which it resides. In many parts of the body, collagen fibres are organized in parallel arrays to form collagen “bundles” as reported by Gunasekaran (U.S. Pat. No. 6,548,077). The glycosaminoglycans and the non-collagenous proteins have to be broken down to extract collagen fibres. The known methods of collagen extraction in fibre form presently practiced all over the world comprises mechanical and physical treatment of tendons followed by enzymatic and chemical processing. No prior art is available at present to extract pure collagen fibres without such treatments.

### OBJECTS OF THE INVENTION

The main object of the present invention is to provide a process for the extraction of atelopeptide collagen from a collagenous source by microbial treatment, which obviates the drawbacks stated above.

Another objective of the present invention is to use a microorganism, which does not produce collagenase.

Yet another objective of the present invention is to use a commercially available strain of Staphylococcal species i.e. Staphylococcus aureus ATCC 29213 having the following characteristics:
1. gram positive, spherical coccus in short chains or clusters.
2. non-motile, non-spore forming, occasionally capsulated.
3. aerobic, ferments sugars, catalase positive and oxidase negative.
4. the organism produces a combination of bound and free catalase, coagulase, hyaluronidase, proteinases, phosphatase, lipase and fibrinolysin-staphylokinase as reported by Mackie & McCartney (Practical Medical Microbiology, Fourteenth Edition, Longman Singapore Publisher, 245-246, 1996).

Yet another objective of the present invention is to use any abundantly available collagenous source.

Still another objective of the present invention is to extract collagen fibres possessing tensile strength as in native tissue, for use as biomaterials.

Yet another objective of the present invention is to extract collagen fibres at neutral pH for use in medical practice.

Yet another objective of the present invention is to knit the fibres into mesh for vascular application, drug delivery and use as haemostatic plug.

### SUMMARY OF THE INVENTION

Accordingly, the present invention provides a process for the extraction of atelopeptide collagen from a collagenous source by microbial treatment, which comprises:
1. treating the said collagenous source with a commercially available strain of Staphylococcus aureus (ATCC 29213), exhibiting characteristics as herein described, in log phase, cultured at neutral pH, for a period in the range of 90-120 hours at a temperature in the range of 20°-40 degree C. and pH between 6.8-8.0 to obtain collagen fibres;
2. treating the collagen fibres, as obtained in step (i), with known disinfectant in the range of minimum 500-3000% (w/v) followed by aqueous washing to obtain decontaminated collagen fibres;
3. drying the decontaminated collagen fibres, as obtained in step (ii), by a known method at a temperature ? 40° C. followed by sterilization by conventional method to obtain atelopeptide collagen.

In an embodiment of the present invention, the collagenous source used may be such as Achilles tendon, extensor tendon.

In another embodiment of the present invention, the staphylococcal species used may be selected from Staphylococcus aureus (ATCC 29213), mixed populations of naturally occurring staphylococcal bacteria, collected from microflora of living organism.

In yet another embodiment of the present invention, the disinfectant used may be selected from the group Consisting of ethanol, petroleum ether, n-propaniol, isopropanol, n-butaniol, isobutanol, 2-methyl propanol, either individually or in any combination.

In still another embodiment of the present invention, the method of drying used may be such as air drying, vacuum drying, dessication.

In yet another embodiment of the present invention, the method of sterilization used may be such as ethylene oxide treatment, gamma ray irradiation, cobalt⁶⁰.

In a further embodiment of the present invention, the period of incubation is between 96-120 hours.

In an embodiment of the present invention, the temperature of incubation ranges from 20 to 40 degree C.

In still another embodiment of the present invention, 5 to 15% w/v of the collagenous source nutrient broth requires 18×10⁷ CFU/ml of Staphylococcus aureus.

In yet another embodiment of the present invention, the pH of the medium of incubation is between 6.8 to 8.0.

### DETAILED DESCRIPTION OF THE INVENTION

The process of the present invention is described below in detail.

Staphylococcus aureus ATCC 29213 is cultured in nutrient broth at neutral pH. Abundant precautions are taken to prevent contamination by maintaining aseptic conditions. A source of collagen rich tissue is thoroughly cleaned and washed several times to remove dirt and blood and placed in the broth containing the log phase of the organism. 5-15% w/v collagenous source: nutrient broth requires 18×10⁷ CFU/ml. The treatment is for 90-120 hours, at a temperature of 20°-40° C. maintaining a pH range of 6.8-8.0 to digest the proteoglycans and the non-collagenous tissues. The collagen fibres left behind in the broth are then transferred into a disinfectant, 500-3000% (w/v), which disinfects the microorganism. The disinfectant is decanted and fresh disinfectant added. This cycle is carried out for a minimum of 2-4 times. Each wash cycle lasts for a minimum of 2-4 hours to ensure complete disinfection. The fibres thus obtained are conventionally washed to remove toxic remains, exo-enzymes and traces of the disinfectant. The decontaminated collagen fibre, thus obtained, is dried by known method at a temperature not exceeding 40° C. and then subjected to conventional sterilization to obtain atelopeptide collagen.

Collagen based biomaterial need to be non-cytotoxic for biomedical applications. Both commercially available collagen as well as collagen derived through microbial treatment posses non-cytotoxic properties. Fibroblast showed same proliferation on commercially available collagen derived through non-inmicrobial treatment (chemical/enzymatic) as well as on collagen derived through microbial treatment.

The inventive step of the present invention lies in identifying the organisms that ensure degradation of non-collagenous components with the help of the protease combination they produce, leaving the collagenous matter intact due to the absence of collagenase in the secreted enzymes, and also in using the same under controlled conditions to extract atelopeptide collagen, which has largely monomeric triple helical conformation.

The following examples are given by way of illustration and therefore should not be construed to limit the scope of the present invention.

### EXAMPLE-1

25 g of Achilles tendon was cleaned of external fatty and adhering tissue and dirt and washed thoroughly with distilled water. 500 ml sterile nutrient broth was inoculated with Staphylococcus aureus ATCC 29213. After 18 hours, the organism attained the log phase 18×10⁷ CFUs/ml. The tendon was placed in the broth containing the organism. Aseptic conditions were maintained to prevent contamination. The temperature was maintained at 37° C. maintaining a pH of 7.0. After 96 hours exposure, the collagen fibres were taken out and disinfected with 50 ml of n-propanol. The disinfectant was decanted and fresh disinfectant added. This cycle was carried out for 3 times. Each wash cycle lasted for 3 hours to ensure complete disinfection. The fibres were later washed in 100 ml of demineralised water at pH 7.0 twice. The fibres were then placed on a nutrient agar plate. No growth of organism was found confirming complete disinfection. The resulting atelopeptide collagen fibres were allowed to air dry at a temperature of 25° C. in a dust free chamber. They were then packed in a polythene sachet and hermetically sealed. The sachets were sterilized using ethylene oxide irradiation for 4 hours.

The atelopeptide collagen prepared in accordance to the above said process was found to possess a crystalline and rope-like structure as revealed by scanning electron microscopy.

The Sodium Dodecyl Sulphate Poly Acrylamide Gel Electrophoresis (SDS-PAGE) of the fibre collagen showed distinct bands of monomeric collagen. The Fourier Transmission Infra Red Spectroscopy also corroborated the retention of helicity of monomeric collagen in the fibre. The Circular Dichroism spectrum revealed more helicity of monomeric collagen in the fibre than the normal soluble collagen fibre prepared by conventional methods.

### EXAMPLE-2

25 g of Achilles tendon was cleaned of external fatty and adhering tissue and dirt and washed thoroughly with distilled water. 500 ml of sterile nutrient broth was inoculated with Staphylococcus aureus ATCC 29213. After 18 hours, the organism attained the log phase 18×10⁷ CFUs/ml. The tendon was placed in the broth containing the organism. Aseptic conditions were maintained to prevent contamination. The temperature was maintained at 37° C. maintaining a pH of 7.0. After 110 hours exposure, the collagen fibres were taken out and disinfected with 50 ml of isopropanol. The disinfectant was decanted and fresh disinfectant added. This cycle was carried out for 4 times. Each wash cycle lasted for 4 hours to ensure complete disinfection. The fibres were later washed in 100 ml of demineralised water at pH 7.0 twice and allowed to air dry in a dust free chamber at a temperature of 25° C. The fibres were then packed wet by placing in a glass tube containing 2 ml of preserving fluid of composition 95% (v/v) isopropanol, 0.6% (v/v) ethylene oxide and 4.4% (v/v) water. The tube was finally hermetically sealed. The sealed tube was again packed in a sachet and sterilized using ethylene oxide irradiation for 4 hours. The atelopeptide collagen fibres were tested for the tensile strength properties on Instron. They were found to possess a tensile strength of 53.42±10.725 MPa.

### EXAMPLE-3

25 g of Achilles tendon was cleaned of external fatty and adhering tissue and dirt and washed thoroughly with distilled water. 500 ml of sterile nutrient broth was inoculated with Staphylococcus aureus ATCC 29213. After 18 hours, the organism attained the log phase 18×10⁷ CFUs/ml. The tendon was placed in the broth containing the organism. Aseptic conditions were maintained to prevent contamination. The temperature was maintained at 37° C. maintaining a pH of 7.0. After 120 hours exposure, the collagen fibres were taken out and disinfected with 50 ml of ethanol-ether (1:1) mixture per gram of collagen. The disinfectant was decanted and fresh disinfectant added. This cycle was carried out for 2 times. Each wash cycle lasted for 3 hours to ensure complete disinfection. The fibres were later washed in 100 ml of demineralised water at pH 7.0 twice and allowed to air dry in a dust free chamber at a temperature of 25° C. The fibres were sterilized using ethylene oxide irradiation for 4 hours. 2.5 gm of fibre collagen and 2.5 gm of commercially available collagen were taken in 1 L of acidified water of pH 2.5-3.0 individually. The pH was adjusted using dilute solution of HCI. After the collagen got solubilised the pH was adjusted to 9.0 by adding dilute NaOH solution. 2% of succinic anhydride solution was prepared in 20 ml of acetone and was gradually added to the collagen suspension. During addition the pH was maintained at 9.0 by adding dilute NaOH solution. Succinylated collagen was precipitated by brining down the pH to 4.2 using dilute HCI. After precipitation, the succinylated collagen was washed repeatedly in water and made to swell in 200 ml of Milli-Q water to form uniform solution. The culture wells were coated with succinylated fibre collagen and succinylated commercially available collagen and ethylene oxide sterilized. Fibroblasts were seeded at a density of 2×10⁴ per well in 24 well microplate. The culture was observed for 5 days. Fibroblast showed same proliferation on commercially available collagen derived through non-microbial treatment (chemical/enzymatic) as well as on collagen derived through microbial treatment.

### EXAMPLE-4

25 g of Achilles tendon was cleaned of external fatty and adhering tissue and dirt and washed thoroughly with distilled water. 500 ml sterile nutrient broth was inoculated with Staphylococcus aureus ATCC 29213. After 18 hours, the organism attained the log phase 18×10⁷ CFUs/ml. The tendon was placed in the broth containing the organism. Aseptic conditions were maintained to prevent contamination. The temperature was maintained at 37° C. maintaining a pH of 7.0. After 100 hours exposure, the collagen fibres were taken out and disinfected with 50 ml of petroleum ether. The disinfectant was decanted and fresh disinfectant added. This cycle was carried out for 3 times. Each wash cycle lasted for 4 hours to ensure complete disinfection. The fibres were later washed in demineralised water at pH 7.0 twice and allowed to air dry in a dust free chamber at a temperature of 25° C. The fibres were packed in a polythene sachet and hermetically sealed. The sachets were sterilized using ethylene oxide irradiation for 4 hours.

In view of the strength and other chemico-physical properties resembling very close to native collagen fibre, gives an added advantage that it can be used effectively for any purpose and perhaps every purpose for which the native collagen fibre is used especially for furthering research concepts and ideas.

### ADVANTAGES

The main advantages of the present invention are the following.
1. It is a single step method to extract intact collagen fibres for biomedical applications.
2. The process saves time, a minimum of 40% over the conventional methods.
3. The method is 60% cost effective due to time saving and other costs involved in conventional methods.
4. The fibres obtained are non-cytotoxic and can be used for medical purposes.
5. The fibres are intact and retain their nativity.
6. The fibres have a tensile strength of 53.42±10.725 MPa.
7. The fibres obtained are at neutral pH.
8. The fibres are crystalline and have a rope-like structure.
9. The process of collagen extraction is devoid of salt formation and hence repeated washing to remove salts and wash water disposal problems do not exist.
10. The fibres obtained can be physically cut with any sharp instrument including a sterile scalpel to suit the dimensions of a-punctual plug.
11. The fibres can be knitted into a mesh for vascular application, drug delivery and use as haemostatic plug.
12. The soluble collagen in solution that can be obtained by solubilising the fibres, has by itself a very high demand in skin care products like creams, shampoo etc. in the cosmetic industry and pharmaceutical industry.

We claim:

1. A process for the extraction of atelopeptide collagen from a collagenotous source by microbial treatment, which comprises:
treating the said collagenous source with a commercially available strain of Staphylococcus aureus (ATCC 29213), exhibiting characteristics as herein described, in log phase, cultured at neutral pH, for a period in the range of 90-120 hours at a temperature in the range of 20°-40 degree C. and pH between 6.8-8.0 to obtain collagen fibres;
treating the collagen fibres, as obtained in step (i), with known disinfectant in the range of minimum 500-3000% (w/v) followed by aqueous washing to obtain decontaminated collagen fibres;
drying the decontaminated collagen fibres, as obtained in step (ii), by a known method at a temperature ? 40° C. followed by sterilization by conventional method to obtain atelopeptide collagen.

2. A process as claimed in claim 1, wherein the collagenous source used is preferably achilles tendon, extensor tendon.

3. A process as claimed in claim 1, wherein the disinfectant used is selected from the group consisting of ethanol, petroleum ether, n-propanol, isopropanol, n-butanol, isobutanol, 2-methyl propanol, either individually or in any combination.

4. A process as claimed in claim 1, wherein the drying of the decontaminated collagen fibers is carried out by air drying, vacuum drying, dessication.

5. A process as claimed in claim 1, wherein the sterilization of the collagen fibres is effected by ethylene oxide treatment, gamma ray irradiation, cobalt⁶⁰.

6. A process as claimed in claim 1, wherein 5-15% w/v of the collagenous source: nutrient broth requires 18×10⁷ CFU/ml of Staphylococcus aureus.

7. A process as claimed in claim 1, wherein the period of incubation is preferably 96 hours.

8. A process as claimed in claim 1, wherein the temperature of incubation is preferably 37 degree C.

9. A process as claimed in claim 1, wherein the preferable pH is 7.2.

10. Use of the strain Staphylococus aureus ATCC 29213 for the extraction of atelopeptide collagen from a collagenous source.
